(19) United States
(12) Patent Application Publication
(10) Pub. No.: US 2024/0074967 A1
(43) Pub. Date: Mar. 7, 2024

(54) METHOD FOR THE TREATMENT OF AN AUDITORY DISEASE OR CONDITION

(71) Applicant: Rinri Therapeutics Limited, Sheffield (GB)

(72) Inventors: Hao LI, Uppsala (SE); Helge Eduard RASK-ANDERSEN, Uppsala (SE); Dan JIANG, London (GB); Gerard Mary O’DONOGHUE, Nottingham (GB); Douglas Edward Hugh HARTLEY, Nottingham (GB); Carlos Marcelo Nicolas RIVOLTA, Sheffield (GB)

(21) Appl. No.: 18/462,678

(22) Filed: Sep. 7, 2023

(30) Foreign Application Priority Data
Sep. 7, 2022 (GB) 2213060.3

(51) Int. Cl. A61K 9/00; A61K 35/12; A61K 45/06

(52) U.S. Cl. CPC A61K 9/0046; A61K 35/12; A61K 45/06

(57) ABSTRACT

A method for the treatment of an auditory disease or condition in a human subject is provided comprising administering a therapeutic agent into a bony cavity of a cochlea of the subject that contains neural structures via the Round Window membrane of the cochlea. Also provided are therapeutic agents for use in such methods, as well as method for the delivery of therapeutic agents via the Round Window membrane of the cochlea.

### CROSS-REFERENCE TO RELATED APPLICATION

This application claims priority to United Kingdom Patent Application No. GB 2213060.3 filed Sep. 7, 2022, the entirety of which is incorporated herein.

The present invention relates to a method for the treatment of an auditory disease or condition.

The focal point of human hearing is the cochlea. This small but complex organ receives sound wave information captured and conducted by the outer and middle ear which is then converted by the cochlea into neural signals that the brain can interpret. This final step of conversion and signalling is handled by specialised sensory cellular apparatus of the cochlea, the hair cells and the auditory neurons. The displacement of the apical stereociliary bundles of the hair cells, produced by the movement of the tectorial membrane, elicits neural potentials which are then relayed to the brain stem via afferent auditory neurons. For sounds to be perceived by the brain, the entire pathway must be intact; any gap or deficit rendering the whole system reduced or ineffective.

The human inner ear contains minute three-dimensional neurosensory structures that are deeply embedded within the skull base, rendering them relatively inaccessible to regenerative therapies for hearing loss. In humans, both types of auditory sensory cell are formed in the developing foetus. These cells lack the capacity for repair or regeneration, therefore those which an individual are born with must last a lifetime. This means that any loss of these cells, or reduction in their performance, is irreversible, resulting in permanent hearing loss.

The structures of the cochlea, such as the basilar and Reissner's membranes, are microscopic and well beyond the resolution of clinical imaging modalities. This is particularly the case of the Rosenthal Canal, a structure that harbours the spiral ganglion neurons in the cochlea. The cell bodies are arranged in a bony helical canal that spirals from the base of the cochlea to its apex; the canal volume is 1.6 μL but with a diffusion potential of 15 μL. The available space is therefore small and difficult to access.

By 2050, deafness due to damage to the sensorineural structures of the inner ear has been predicted to affect 2.5 billion individuals globally with major health, economic and societal implications. Yet no biological treatment exists for this disabling condition. Auditory prostheses, such as hearing aids and cochlear implants, represent the mainstay of current management but have considerable functional limitations. These devices bypass the damaged inner ear hair cells by converting signals into electric pulses that directly stimulate the auditory neurons which are transmitted to the brain and heard as sound. The greatest challenge today is to find a curative treatment for hearing loss through restoration of the neurosensory substrates that underpin the ability to hear. However, when there is damage to the auditory neurons, as in presbycusis and Auditory Neuropathy Spectrum Disorder (ANSD), poor innervation severely limits the prospective performance of the CI and, subsequently, any benefit that a patient derives from that device may be limited. The challenge is formidable as the human cochlea is a highly specialised post-mitotic organ with highly restricted proliferative and regenerative capabilities.

Over 90% of disabling hearing loss is sensorineural in origin, meaning the specialist cells of the cochlear that convert, transmit and process sound are damaged or dead. Whilst all causes of sensorineural hearing loss are associated with damage to the cells that detect and amplify sound (hair cells), or damage to the cells that transmit the sounds to the brain (auditory nerves), or both, presbycusis and ANSD are conditions that are both associated with auditory nerve damage and/or dysfunction.

The cochlea's sensory receptors comprise vibration-sensitive hair cells, their synapses and associated neurones. The system is finely tuned and can respond within millionths of a second to displacement of atomic dimensions. The neurosensory structures are fragile and may be permanently lost due to genetic and environmental factors and are particularly susceptible to the ageing process. The hearing loss may be due to a loss of hair cells (Hudspeth, A. J., Nat. Rev. Neurosci. 2014 159 15, 600-614 (2014)), synaptic dysfunction (Liberman, M. C. & Kujawa, S. G., Hear. Res. 349, 138-147 (2017) or to depletion of the neural population within the cochlea (Zhang et al., Ear Hear. 35, 410-417 (2014)).

The alarming rise in prevalence of hearing loss, now affecting 7% of the world's population, is a major stimulus to exploit the potential of regenerative medicine in this field (Wilson et al., Lancet (London, England) 390, 2503-2515 (2017)). Hampering progress has been the inaccessibility of the human cochlea which lies in the skull base deeply encased in the hardest bone in the human body. Yet, once accessed, the cochlea promises to be a receptive organ for neurosensory regeneration: the neurosensory cells are relatively few in number and its minute fluid compartments (with a total volume around 200 μL) are tightly confined with a negligible circulation which should facilitate biological effectiveness and restricted biodistribution, minimising off-target effects. In addition, cochlear tissues are relatively immune-privileged being protected by the blood-labyrinth barrier thus dampening the inflammatory rejection process. Recent elaboration of a range of molecular mechanisms responsible for inner ear dysfunction have opened a vista of opportunities for a range of novel therapeutic approaches to hearing loss including small molecules, gene and cell therapies (Korver, A. M. H. et al. Nat. Rev. Dis. Prim. 3, (2017)).

Central to success, however, is the ability to deliver therapeutic agents safely and precisely to their target structures within the relatively impenetrable human cochlea. Prior speculation about administration of stem cells into the inner ear has not enabled such techniques to be performed safely (or to be considered safe to perform) in human subjects. Until now, surgical access has not been available to the spaces that harbour the neural structures of the cochlea, for instance Rosenthal's canal or the Canalis cochlearis within the central core of the human cochlea. However, the present invention provides a method for safely and effectively accessing Rosenthal's Canal or the Canalis cochlearis for regenerative therapeutic interventions in humans.

According to a first aspect of the invention there is provided a method for the treatment of an auditory disease or condition in a human subject, comprising administering a therapeutic agent into a bony cavity of a cochlea of the subject that contains neural structures via the Round Window membrane of the cochlea.

The bony cavity of the cochlea of the subject that contains neural structures may be either the Rosenthal's Canal or the Canalis cochlearis. The term Canalis cochlearis as used in the context of the present invention is used to refer to the canal that contains the cochlear nerve, not the cochlear membranous labyrinth (or duct). In the present disclosure, the term Canalis cochlearis does not therefore include reference to the cochlear duct which is properly known as the Ductus cochlearis. The term Canalis cochlearis as used herein therefore excludes the cochlear membranous labyrinth (or duct) known as the Ductus cochlearis. The term Canalis cochlearis as used herein therefore refers to the canal containing the cochlear nerve and excludes the cochlear membranous labyrinth.

The method of the invention therefore does not target the scalae timpani and vestibuli, two of the perilymph-filled compartments of the cochlea.

The method of the invention does not include any approach to the auditory nerve via an intracranial surgical procedure such as a retrosigmoid craniotomy normally used for tumour removal.

Prior to the making of the present invention, surgical access to the cochlea (the organ of hearing also known as the “inner ear”) has largely been restricted to entering one of the fluid-filled chambers that transmits sound vibrations through the ear called the “scala tympani”, often via a small membrane that separates it from the middle ear called the “Round Window”. These sound vibrations are converted into nerve impulses through deflection of hair cells within the cochlea that are heard in the brain as sound. The present invention provides a novel, safe, effective, and minimally invasive surgical approach to the central core of the inner ear that contains the hearing nerve cells.

Auditory diseases or conditions, for example profound deafness, are often associated with a significant loss of hair cells and are currently treated with a hearing device called a cochlear implant. A cochlear implant converts sounds into electrical pulses that stimulate the nerve fibres within the cochlea via electrodes that are surgically implanted within the scala tympani. The nerve fibres are located in bony chambers within the central core of the inner ear.

The nerve fibers closest to the hair cells are contained within a bony chamber that spirals around the central core of the inner ear called “Rosenthal's Canal”. These nerve fibers exit the inner ear and form a nerve bundle within a larger bony chamber called the “Internal Auditory Canal” (IAC) that contains cerebrospinal fluid (“CSF”). This bundle of hearing nerves connects to base part of the brain also referred to as the “brainstem”. The Canalis cochlearis is the part of the Internal Auditory Canal that is closest to the cochlea.

The administration of the therapeutic agent to the Rosenthal's Canal or the Canalis cochlearis may be via the Round Window membrane of the cochlea. The auditory disease or condition may be a hearing loss (i.e., sensorineural hearing loss), deafness, or other auditory disorder associated with loss of inner ear function. The auditory disease or condition may have an environmental or genetic aetiology. The auditory disease or condition may be cancer, a bacterial infection or an inflammatory disease or condition. The broad term sensorineural hearing loss (SNHL) includes loss of/damage to both auditory hair and neuron cells. Age-related hearing loss (presbycusis) and Auditory neuropathy spectrum disorder (ANSD) are subsets of sensorineural hearing loss, that are associated with loss and/or abnormal function of auditory neurons. In one embodiment, the methods of the invention are for the treatment of hearing loss characterised as neural loss and/or dysfunction associated with presbycusis and auditory neuropathy spectrum neuropathy disorder (ANSD).

Syndromic and non-syndromic disorders cause ANSD through their effect on synaptic and neural pathways. ANSD varies by aetiology and includes two main subtypes that are characterised by either a defective i) connection between the hair cells and neurons (synaptopathy), or ii) neural conduction of the signal to the brain (auditory neuropathy). Individuals with synaptopathy are known to perform better with a cochlear implant, compared with individuals with auditory neuropathy, since the cochlear implants is able to bypass the site of the lesion.

A cell-based therapy for hearing loss based on administration of stem cells to replace auditory neurons may benefit individuals with auditory neuropathy. In other individuals with synaptopathies, particularly those arising from deficits on the side of the inner hair cells (pre-synaptically), the treatment regime may involve implantation of a cochlear implant as well as a cell-based therapy according to the methods of the present invention.

Genes that are known to cause ANSD arising from synaptopathy include i) OTOF, CACNA1D, CABP2 and SLC17A8 that all cause pre-synaptic deficits on the side of the inner hair cells, and ii) DIAPH3, OPA1, ROR1 and ATP1A3 that all cause post-synaptic deficits on the side of the spiral ganglion neurons. Genes that are known to cause ANSD arising from auditory neuropathy include TIMM8A, AIFM1, NARS2, MPZ and PMP22. Methods of the present invention may therefore be directed to the treatment of a genetic disease characterised by one or more of the genes described above.

Other therapeutics targets suitable for treatment, include but are not limited to hearing loss associated with:
infections (e.g., lues, mumps)
ototoxic drug exposure (exposure to drugs or chemicals that damage the inner ear)
immune disorders (Guillain-Barre, etc)
neurological disorders
neoplasms (e.g., acoustic neuroma, brainstem meningiomas)
non-syndromic dominant autosomal, recessive autosomal, X-linked, or mitochondrial genetic conditions and other hereditary neuropathies e.g., Friedreich ataxia and Charcot-Marie-Tooth disease, Leber's Hereditary Optic Neuropathy, Deafness-Dystonia-Optic Neuronopathy (DDON) syndrome (Mohr-Tranebjaerg syndrome)

Additionally in a neonatal subject other therapeutic targets suitable for treatment are hearing loss associated with:
premature birth
lack of oxygen (anoxia) at birth
hyperbilirubinemia, possibly requiring blood transfusion, associated with severe
jaundice during the newborn period
prolonged neonatal intensive care unit (NICU) stay

The therapeutic agent may be a pharmaceutical composition, for example a composition comprising a pharmaceutically active substance, or for example a composition comprising a population of cells, such as stem cells or auditory or sensory neurons. The pharmaceutically active substance may be a compound, growth factor, hormone, or specific binding molecule, such as an antibody or fragment thereof.

In one embodiment, the method of this aspect of the invention may comprise administering a therapeutic agent into the Rosenthal's Canal in a cochlea of the subject via the Round Window membrane of the cochlea.

In an alternative embodiment, the method of this aspect of the invention may comprise administering a therapeutic agent into the Canalis cochlearis in a cochlea of the subject via the Round Window membrane of the cochlea.

The Rosenthal's Canal and Canalis cochlearis are bony cavities that contain neural structures which define spaces that can be safely and effectively accessed for delivery of therapeutic agents according to a method of the present invention. The Canalis cochlearis is the part of the Internal Auditory Canal that is closest to the cochlea.

The auditory nerve is located in the centre of the cochlea and is comprised of auditory neurons. The cell bodies of these auditory neurons reside in discrete compartments termed Rosenthal's Canal. The Round Window membrane is located at the external or distal end of the Rosenthal's Canal with respect to the interior of the cochlea. The Canalis cochlearis houses the main branches of the cochlear nerve. This relatively large space is housed within the pouch of subarachnoid nerve components including nerve fascicles already separated reaching the cochlear frequency areas at 1 kHz and below as well as the nerve trunk containing axons tuned for frequencies below 1 kHz.

The term antibody or fragment thereof includes such molecules as a full length (IgG) immunoglobulin molecule as well as, for example, a Fab, F(ab′)₂, Fab₂, bispecific Fab₂, trispecific Fab₂, Monovalent IgG, Fc, scFv, bispecific diabody, trispecific triabody, scFv-Fc, minibody, hcIgG or VhH molecule, as well as a fusion protein of such a fragment with another therapeutically active protein or fragment thereof.

The compound may be an activator of inner ear progenitor cells or other molecule capable of stimulating inner ear progenitor cell growth, such as FX-322 and/or FX-345, or the compound may be an anticancer agent, an otoprotective agent, an antibiotic, or an anti-inflammatory agent (such as a steroid or non-steroidal anti-inflammatory agent). The growth factor may be selected from the group consisting of insulin-like growth factor-1 (IGF-1), a neurotrophin (such as nerve growth factor (NGF), brain-derived neurotrophic factor (BDNF), neurotrophin-3 or neurotrophin-4) or a steroid (such as dehydroepiandrosterone or dehydroepiandrosterone sulfate).

The population of cells may comprise fully- or partly-differentiated cells. The fully- or partly-differentiated cells may be derived from stem cells. Examples of fully differentiated cells may include but are not limited to auditory neurons, hair cell-like cells, spiral ganglion neurons or sensory neurons. Alternatively, the population of cells may comprise stem cells (such as mesenchymal stem cells, embryonic stem (ES) cells, induced-pluripotent stem (iPS) cells or parthenote-derived stem cells) or progenitor cells (for example otic progenitor cells, such as otic neural progenitor cells (ONPs)). The progenitor cells may be derived from stem cells, e.g., ES cells, such as human embryonic stem cells (hESCs) or human induced pluripotent stem cells. Suitable methods of preparing ONPs are described in WO 2016/156831 and WO 2018/051092.

In the case where the therapeutic agent is a population of cells, the agent may also be administered with one or more other pharmaceutically active agents as described herein. Suitably, in such embodiments, the population of cells may be present in a composition comprising one or more other pharmaceutically active agents as described herein.

Where the therapeutic agent is a population of cells, the cells may be derived from the subject to be treated, i.e., autologous, or the cells may be engineered to be syngeneic or otherwise immunologically compatible with the subject. Alternatively, the cells may be allogeneic or xenogeneic. For cells which are immunologically incompatible, it may be desirable to co-administer immunosuppressive agents to the subject to be treated also.

Suitably, the site of administration of the therapeutic agent into the Rosenthal's Canal may be 3-4 mm from the Round Window membrane of the human cochlea. The administration into the Rosenthal's Canal may be through the superior mid-region of the Round Window membrane. The administration into the Canalis cochlearis may be medial to the inferior cochlear vein and may pass anterior to the cochlear aqueduct.

Suitably, the contents of the Rosenthal's Canal may be 3 to 4 mm deep with respect to the Round Window membrane. The administration into the Rosenthal's Canal may be through the superior mid-region of the Round Window membrane. The administration into the Canalis cochlearis may be medial to the inferior cochlear vein and may pass anterior to the cochlear aqueduct.

After reflecting back the Round Window membrane, the Crista fenestra may be used as a surgical landmark for drilling at the junction between the floor of the scala tympani and wall of the modiolus to access the Canalis cochlearis. The surgical hole created is therefore medial to the inferior cochlear vein.

Administration of the therapeutic agent according to the methods of the invention is therefore by means of a surgical intervention. The surgical intervention comprises the injection of the therapeutic agent into a bony cavity of a cochlea that contains neural structures of a subject, for example into the Rosenthal's Canal or Canalis cochlearis, via the Round Window membrane of a cochlea of a human subject. The injection may be suitably made after preparing an image of a cochlea of the human subject, such as by using High Resolution Computerized Tomography (CT) or Cone beam CT in combination with a navigation system (but such imaging is not essential).

Following the present invention and as described herein, it has now been demonstrated that 3-dimensional models based on imaging data, such as by SR-PCI data, allow the conception of highly accurate intervention pathways which have been validated by anatomical dissection and micro-radiographic imaging such as X-ray phase-contrast imaging (PCI), such as synchrotron radiation phase-contrast imaging (SR-PCI). The findings described herein present a novel, safe and effective surgical access to bony cavities within the cochlea that contain the neural structures such as the Rosenthal's Canal or Canalis cochlearis that will de-risk future clinical interventions and pave the way for clinical trials of therapeutic agents for the treatment of auditory diseases or conditions. The discovery that the Rosenthal's Canal or Canalis cochlearis can be targeted and used to administer therapeutic agents to the human cochlea therefore represents a significant breakthrough in the treatment of auditory diseases and conditions.

This aspect of the invention extends to a therapeutic agent for use in a method for the treatment of an auditory disease or condition in a human subject, wherein the therapeutic agent is for administration into a bony cavity of a cochlea of the subject, for example the Rosenthal's Canal or Canalis cochlearis, via the Round Window membrane of the cochlea.

In some embodiments, the invention extends to a therapeutic agent for use in the manufacture of medicament for the treatment of an auditory disease or condition in a human subject, wherein the therapeutic agent is for administration into a bony cavity of a cochlea of the subject, for example the Rosenthal's Canal or Canalis cochlearis, via the Round Window membrane in the cochlea.

Formulations and dosages suitable for administration according to a method of the present invention can be determined by the medical practitioner according to the nature of the disease to be treated and the therapeutic agent to be delivered.

The therapeutic agent for administration according to a method of the invention may be in a volume of from 10 μl to 30 μl, suitably 15 μl to 25 μl, such as for example 15 μl, 16 μl, 17 μl, 18 μl, 19 μl, 20 μl, 21 μl, 22 μl, 23 μl, 24 μl, or 25 μl. The therapeutic agent may be formulated in an appropriate aqueous or non-aqueous medium, suitably including for an aqueous medium where appropriate a physiologically acceptable diluent and/or buffer.

Patients receiving a cell-based therapy may receive a dosage of 0.1E+06 to 2.0E+06 cells/ear, for example 0.15E+06 cells/ear. Suitably, the dosage may be administered as a single dose suspension.

According to a second aspect of the invention, there is provided a method for the delivery of a therapeutic agent to a cochlea of a human subject having an auditory disease or condition, comprising administering the therapeutic agent into a bony cavity of the cochlea of the subject via the Round Window membrane of the cochlea.

Suitably, the contents of Rosenthal's Canal may be 3 to 4 mm deep with respect to the Round Window membrane. The administration into the Rosenthal's Canal may be through the superior mid-region of the Round Window membrane. The administration into the Canalis Cochlearis may be medial to the inferior cochlear vein and may pass anterior to the cochlear aqueduct.

The present invention provides for the first time a new delivery route direct into a cavity of a cochlea of the subject that contains neural structures, for example into the Rosenthal's Canal or Canalis cochlearis of the human cochlea. While injection into the central core of the human inner ear, i.e., a cavity of a cochlea (that includes Rosenthal's Canal) has been speculated about, the complexity and relatively small size has prevented the identification of suitable routes for delivery of therapeutic substances into these spaces.

The novel surgical approach described herein for delivery of therapeutic agents to the central core of the cochlea that contains the hearing nerve fibers, including Rosenthal's Canal and the Internal Auditory Canal containing the Canalis cochlearis, was developed based on computer modelling and experiments on deceased human skull specimens. Together this work showed that surgical access to the bony chambers that contain the hearing nerves within the cochlea was both safe and effective, including Rosenthal's Canal and the Internal Auditory Canal, via the Round Window.

Although surgical entry into the cochlea via the Round Window is a standard surgical approach that is currently used during the insertion of a cochlear implant, the present inventors have showed that it is possible to consistently access these structures with a needle, without damaging any of the surrounding structures including any blood vessels, other nerves, or any of the other delicate structures within the cochlea as in a standard surgical approach as used in cochlea implant surgery.

Whilst the present disclosure describes two new surgical approaches to both Rosenthal's Canal and the Canalis cochlearis of the Internal Auditory Canal, the surgical approach to the internal auditory canal may be preferred since it provides greater access to a larger number of hearing nerve fibers, and permits injection of larger volumes, compared with the surgical approach to Rosenthal's Canal. This conclusion was supported by imaging experiments that involved injection of a fluid that shows up on scans.

Whilst other surgical approaches to the internal auditory canal have been previously been described, all these approaches, including rtranspetrous' routes (e.g., ‘translabyrinthine’, ‘transotic’, ‘transcochlear’), the ‘retrosigmoid’ route, and the ‘middle cranial fossa’ techniques. All of these procedures are usually used for removal of tumors and are highly invasive due to the extensive bone work, the brain or cerebellar retraction, or the facial nerve re-routing to reach the affected area. Unlike these approaches, for the first time the present inventors have described a minimally invasive approach via the Round Window into internal auditory canal and Rosenthal's Canal that contain hearing nerve fibers. Critically both of these approaches are associated with preservation of all the delicate structures within the cochlea.

Preferred features of the second and subsequent aspects of the invention are as for the first aspect of the invention mutatis mutandis.

The present invention will now be described by way of illustration with reference to the following Examples and Drawings which are not to be construed as being limitations on the invention.

### BRIEF DESCRIPTION OF THE DRAWINGS

FIG. 1 shows (a) Synchrotron phase contrast imaging (SR-PCI) with 3D orthographic rendering of an intact human inner ear. The bony wall of the cochlea was made semi-transparent to permit visualization of the basilar membrane (BM), Rosenthal's canal (RC) and auditory nerve (AN). The auditory nerve contains approx. 30,000 fibres and their cell bodies are located in a 14.5 mm long spiral bony canal called RC. From there, peripheral neurites spread out to innervate approx. 15 000 hair cells placed on the BM. A probe is shown penetrating the round window (RW) membrane to access the underlying RC.
(b) Microradiograph taken following placement of a radio-opaque marker at the presumed site of RC on anatomical dissection; the image confirms precise targeting of RC during dissection.

FIG. 2 shows (a) The surgical approach to Rosenthal's Canal (RC) passing through the mastoid bone behind the ear. Note the structures of surgical interest: the facial nerve (FN), the stapes (S) and stapedius muscle (SM) and round window membrane (RVV). A surgical trephine is seen penetrating the RW to reach RC located just deep to it.
(b) A 4×4 dynamic grid was applied to 10 RW membranes and was adjusted to membrane size for each specimen.
(c) X denotes those grid units in closest anatomical proximity to RC in which a given penetration had at least an 80% chance of reaching RC.
(d) Using this data, a heat map was created for optimal targeting of RC behind the RW membrane. Highest probability of targeting the RC was through the superior mid-region of the RW membrane. At A2 and A3, the chance was 90% and 80% respectively. Higher values were noted in C1 and B1 (100%), but this region risks injury to the vestibulo-cochlear artery. *D1 was covered by stapedius muscle and was thus not evaluable. Bar at the bottom displays the actual frequencies of successfully targeting the RC.

FIG. 3 shows exposed the medial wall of the modiolus and the under surface of the spiral lamina. Site of injections is shown as a white arrow in FIGS. 3(a), (c) and (d). Also shown in FIG. 3(b) is an orthogonal section showing the 3D reconstruction and direction of injection needle to reach the internal acoustic meatus and cochlear canal. The distance is here only less than 1 mm. The spread nerve fibers enter the bony perforation to the basal turn can be seen.

FIG. 4 shows 2D images of material injected the course of temporal bone studies (bone 466001). The upper two images in (a) and (b) show a drilled well and metal wire reaching into the IAC. The lower two images in (c) and (d) show the previous “dark nerve tissues” in the IAC became “white” after injection.

### EXAMPLES

### Example 1: Identification of the Rosenthal's Canal as Route for Administration into Human Cochlea

Synchrotron radiation phase-contrast imaging (SR-PCI) was used to delineate the minute structures of the cochlea while leaving them in situ. SR-PCI differs from conventional radiography in being able to allow a phase shifted beam to interact with the original beam to produce fringes that represent the structural and surface boundaries (edge enhancement) of a specimen. This phase-contrast imaging produces images with excellent soft-tissue and bone discrimination. For the first time it has been possible to accurately image the detailed cytoarchitecture of the human inner ear in the intact state without incurring artefacts that so compromised previous anatomical studies. The use of advanced computer vision tools enabled fine blood vessels to be imaged and the pathways of nerves to be tracked through to the bony central core (or modiolus) of the cochlea. This level of detail subsequently informed the planning of a safe and effective surgical approach for clinical application.

Rosenthal's canal (RC), which houses the cell bodies of the 30 to 35,000 human spiral ganglion (HSG) neurons of the auditory nerve, hugs the modiolus and extends from its base to near the cochlear apex (Figurel). The physical characteristics of RC are described in Table 1. The analysis described herein determined that it averages 14.57 mm in length (range 14.02-15.08 mm) and that it is covered by bone with a thickness of 28-56 μm which may be deficient in parts. The diameter of RC varies from 0.1 mm to 0.5 mm, being at its greatest towards the apex of the helix and has an average volume of 1.6 μL. The central projections of the auditory nerve traverse a virtual space containing cerebrospinal fluid as they exit the cochlea on their way to the brain-stem. It was calculated that this space has a potential volume of 15 μL and as these nerve fibres are devoid of perineurium and may thus be receptive to cell or gene-based therapies.

At the base of the cochlea the RC lies in close proximity to the round window membrane (FIG. 1), a structure that is easily identifiable during routine ear surgery and that can be readily penetrated or reflected to gain access to RC. The SR-PCI 3-dimensional models were used to define the round window to RC relationships and then undertook a series of surgical simulations, formulating a heat map to define the surgical trajectory that would lead directly to RC with the highest probability and without damage to adjacent structures (FIG. 2). It was found that by using a dynamic grid adjusted to the size of the round window that siting the point of penetration superiorly would reach target in 80% of cases without compromising cochlear blood supply which runs immediately adjacent to RC. Depending on the trajectory used, the contents of RC lay just 3 to 4 mm deep to the Round Window. Furthermore, to facilitate clinical translation, the trajectories were ensured to be consistent with instrumentation and approaches compatible with those used in routine ear surgery.

In order to confirm the validity of the proposed route of access, a series of surgical studies were carried out on human temporal bones. The aim was to determine if RC could be reached using surgical approaches through the mastoid bone with standard instrumentation. Six temporal bones were used, with surgeons marking the expected site of RC by positioning a radio-opaque metallic marker. The specimens were scanned prior to dissection (to exclude structural anomalies) as well as following placement of the marker. The bones were scanned with micro-computed tomography, following a protocol described elsewhere 9. In 5 of the 6 temporal bones, the marker was either within the RC or immediately adjacent to it—results that were in line with those predicted from the modelling data; in one bone the marker ended up fractionally below target. In none of the temporal bones was the microvasculature disrupted and no unintended damage to other anatomical structures within the ear was observed.

The inability to adequately image the fine structures of the human inner ear has been a major barrier to advance therapies for this complex end-organ. SR-PCI is proving to be transformative in displaying and evaluating these microscopic structures providing unprecedented visualisation of its in-situ cytoarchitecture. Traditional methods used to design surgical routes to the cochlea, even with operating microscopes, were marred by their inherent destructive nature. It has now been demonstrated that 3-dimensional models based on SR-PCI data allow the conception of highly accurate intervention pathways which were subsequently validated by surgical studies and micro-radiographic imaging. The findings described herein will de-risk future clinical interventions and pave the way for clinical trials of novel inner ear therapies for the treatment of hearing loss. The application of SR-PCI to the auditory system also dovetails with an escalation of interest in regenerative inner ear therapies which hold considerable promise for addressing the growing health burden of hearing loss (Andres-Mateos, E. et al. Nat. Commun. 13, 1359 (2022); Lustig, L. & Akil, O. Cold Spring Harb. Perspect. Med. 9, (2019). Crane et al., Front. Neurosci. 15, (2021)). While considerable challenges remain in developing novel therapeutics for use in humans (Plontke, S. K. & Salt, A. N. Hear. Res. 368, 1-2 (2018)), it is believed that the methods described herein herald a new era for the application of regenerative therapeutics to the inner ear.

Material and Methods

Ten adult human temporal bones were obtained with permission from the Body Bequeathal Program at Western University, London, Ontario, Canada in accordance with and approved by the Anatomy Act of Ontario and Western University's Committee for Cadaveric Use in Research (approval #19062014).

The imaging technique used in this study is the propagation-based X-ray phase-contrast imaging (PCI) method, which is also known as in-line PC and has previously been used to image the auditory system (Elfarnawany, M. et al. J. Microsc. 265, 349-357 (2017); Koch et al., J Otolaryngol Head Neck Surg 46, 19 (2017); Mei, X. et al. Sci. Rep. (2020) doi:10.1038/s41598-020-62653-0). Compared to conventional X-ray absorption-based imaging, in-line PCI uses X-ray refraction which highlights tissue boundaries within a sample. It can be used to image soft tissues which do not absorb X-rays sufficiently to distinguish tissue components based on image contrast. A spatially coherent source is needed for in-line PCI, hence synchrotron radiation is used in this work rather than a conventional X-ray source. The overall set-up for in-line PCI is similar to typical absorption-based radiography in that it consists of a source, a sample, and a detector; however, the main difference is that the detector is placed further from the sample when using in-line PCI, and this gives rise to Fresnel fringes. In-line PCI is sensitive to changes in refractive index which leads to edge enhancement in images.

SR-PCI scanning was performed at the Bio-Medical Imaging and Therapy (BMIT) 05ID-2 beamline at the Canadian Light Source Inc. located in Saskatoon, SK, Canada. The X-ray photon energy was 42 keV, with sample-to-source distance of 57 meter and sample-to-detector distance of 2 m. The detector had field of view of 36 mm×9.5 mm and pixel size of 9 μm, and 3000 projections were collected over 180° rotation. The reconstruction was performed using the UFO platform (www.github.com/ufo-kit), which is an open-source platform.

To perform the quantitative analysis and 3D visualization, phase-retrieval technique was used to convert the edge enhancement caused by fringes, to areal contrast using Paganin/TIE method (Paganin et al., J. Microsc. 206, 33-40 (2002)). The reconstructed slices where then imported to 3D Slicer (www.slicer.org) for visualization, segmentation and measurements (as described above). Manual threshold painting was performed for most anatomical structures. Measurements of volumes and distances to adjacent critical structures was then undertaken and trajectory maps for future surgical approaches were designed. Image segmentation was driven by the need to survey the anatomical structures of clinical interest. Semi-automatic and manual segmentation tools, threshold painting, thresholding, tractography, and scissors tools were used to display the fine detail of the structures of interest.

TABLE 1

| | RC length (mm) | RC diameter (mm) | | | | RC volume (uL) |
|---|---|---|---|---|---|---|
| Bone # | Total | RC base | 16000 Hz | 45° | 90° | Total |
| 1 | 14.02 | 0.133 | 0.352 | 0.291 | 0.435 | 1.182 |
| 2 | 14.73 | 0.100 | 0.189 | 0.366 | 0.498 | 1.376 |
| 3 | 14.28 | 0.109 | 0.158 | 0.395 | 0.464 | 1.087 |
| 4 | 14.61 | 0.060 | 0.150 | 0.442 | 0.430 | 1.392 |
| 5 | 14.22 | 0.116 | 0.333 | 0.432 | 0.495 | 1.668 |
| 6 | 15.08 | 0.143 | 0.449 | 0.382 | 0.464 | 1.746 |
| 7 | 14.91 | 0.103 | 0.246 | 0.482 | 0.590 | 1.246 |
| 8 | 14.58 | 0.109 | 0.246 | 0.494 | 0.567 | 2.315 |
| 9 | 14.27 | 0.122 | 0.247 | 0.468 | 0.444 | 2.012 |
| 10 | 14.98 | 0.096 | 0.270 | 0.519 | 0.637 | 2.150 |
| Average | 14.568 | 0.109 | 0.264 | 0.427 | 0.502 | 1.617 |
| SD | 0.36058 | 0.023 | 0.093 | 0.069 | 0.072 | 0.430 |

Table 1 shows the length, diameter and volume of Rosenthal's Canal (RC) as determined by synchrotron phase-contrast imaging in the 10 human temporal bones in the study. The diameter of the RC is larger with increasing distance from the base of the cochlea.

### Example 2: Identification of Canalis cochlearis as Route for Administration into the Human Cochlea

The route of administration of a therapeutic agent into the Canalis cochlearis (cochlear canal) was discovered as follows. The imaging of the cochlear and construction of a three-dimensional model was as described above in relation to Example 1.

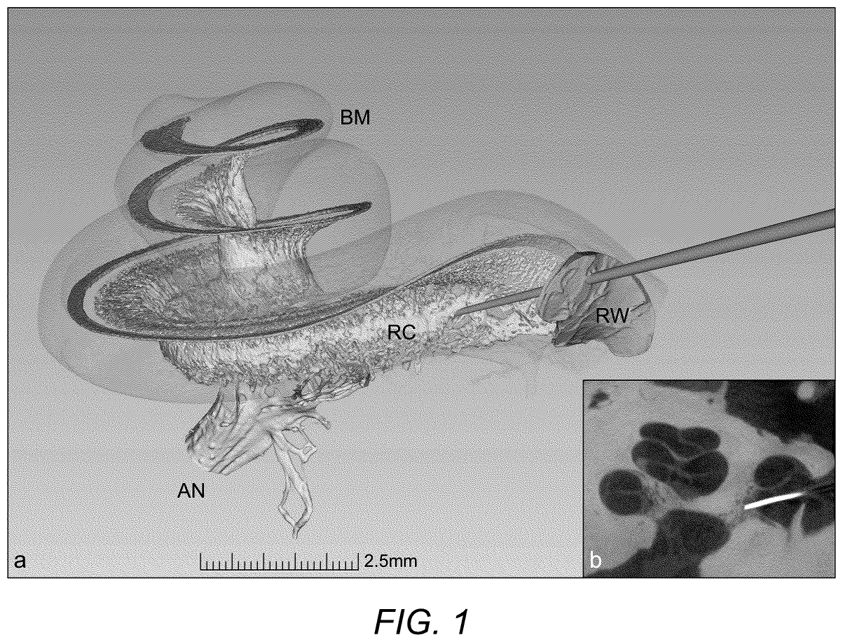

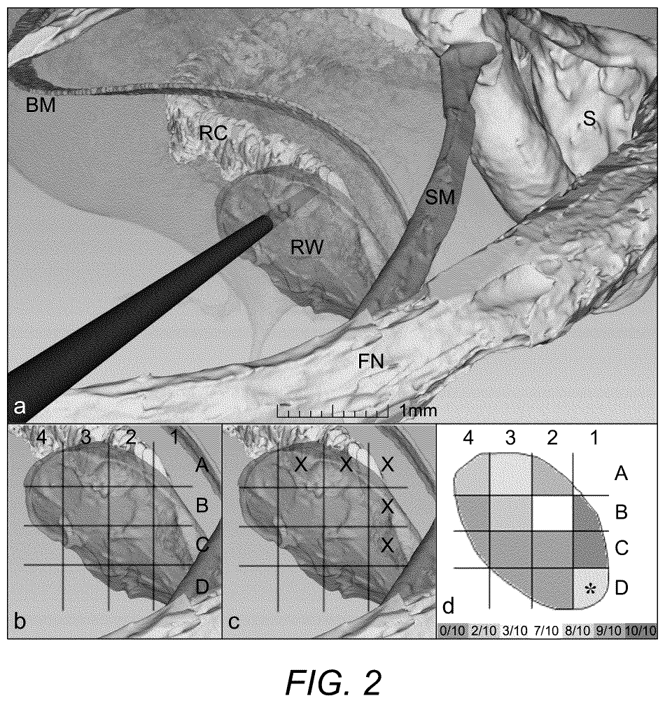

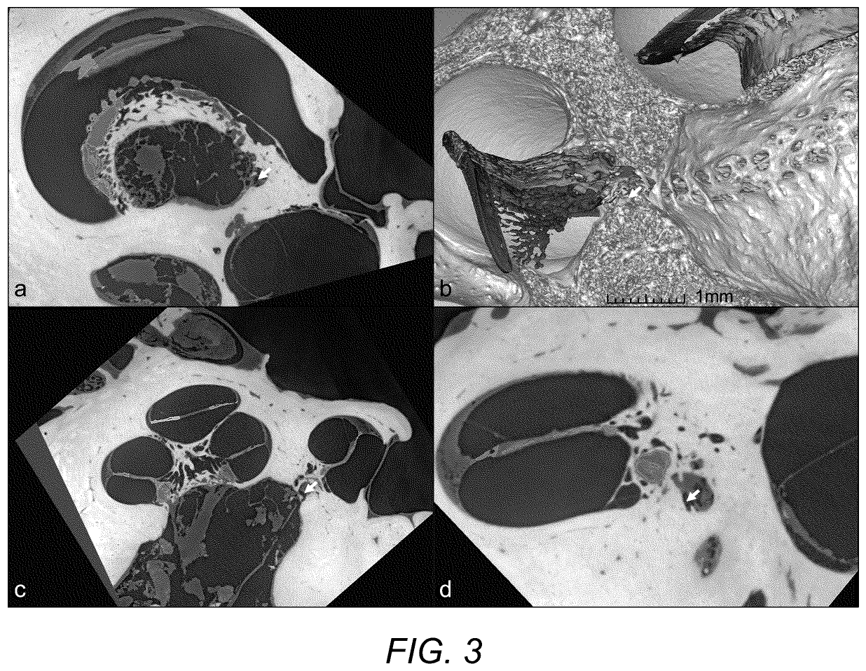

The Round Window membrane was entirely exposed through a surgical posterior tympanotomy, Next, the cochlear aqueduct and Crista fenestra were identified at the anterior inferior rim of the Round Window membrane. Subsequently, the Round Window membrane was loosed and lifted upwards. See FIG. 3. Crista fenestra and chorda tympani were used as clinical reference points. During straight drilling approach, the drill is placed vertically next to Crista fenestra In the angled drilling approach, the drill is placed next to the Crista fenestra, but leaning closer towards chorda tympani.

Observations were made regarding drills reaching to the Canalis cochlearis. Drilling distance between Crista fenestra and Canalis cochlearis were measured, together with effective bone thickness from behind crista to Canalis cochlearis.

TABLE 2

| | Angled close | | | | | |
|---|---|---|---|---|---|---|
| | to chorda | | | Straight | | |
| | mm | | | mm | | |
| | Crista to | | | Crista to | | |
| | Canalis | mm | Touch | Canalis | mm | Touch |
| Bone # | Cochlearis | Bone | Vein | Cochlearis | Bone | Vein |
| 1 | 2.822 | 2.046 | x | 2.571 | 1.683 | x |
| 2 | 3.35 | 2.523 | | 3.025 | 2.062 | x |
| 3 | 3.091 | 2.197 | | 2.878 | 1.807 | |
| 4 | 2.082 | 1.821 | | 2.268 | 1.672 | |
| 5 | 4.525 | 3.716 | | 3.716 | 1.277 | |
| 6 | 6.237 | 4.887 | unclear | N/R | N/R | unclear |
| 7 | 2.705 | 2.233 | | 2.2 | 1.64 | |
| 8 | N/R | N/R | | 3.65 | 2.88 | |
| 9 | 1.595 | 1.008 | | 2.478 | 0.688 | |
| 10 | 2.46 | 1.747 | | 2.572 | 1.602 | |
| average | 3.20744444 | 2.464222 | | 2.81755556 | 1.701222 | |
| SD | 1.40420503 | 1.16129 | | 0.55527293 | 0.58654 | |

Table 2 shows the distance between Crista fenestra and drill tip reaching into Canalis cochlearis were measured, as well as drilling distance in the bone were measured in 10 bones. (Key to symbols on table: X is drill touched inferior cochlear vein; N/R is not reached Canalis cochlearis).

Both approaches (straight and angled drilling) resulted in 9/10 reaching the Canalis cochlearis. In the straight approach, drills touched the inferior cochlear vein in 2 bones. In the angled approach group, drills touched same vein in one bone. Due to unclear image, visualizing inferior cochlear vein in one of the bones was not possible. Average drilling distance is larger in the angled approach group, 2.5 mm compared to 1.7 mm in the straight drilling group. Standard deviation higher as well, probably due to anatomical variances of the chorda tympani and size of facial recess.

### Example 3: Injection of Otic Progenitor Cells into the Canalis cochlearis of the Internal Auditory Canal (IAC) or Rosenthal's Canal (RC)

Temporal bones were operated and a modiolar well was made behind the Round Window according to the methods described herein. A metal wire was inserted into the modiolar well to simulate the injection needle path and subsequently a risk analysis was made with regard to the nearby facial nerve and vasculature. After removing the metal wire, otic progenitor cells mixed with contrast medium potassium iodide (KI) were injected into the IAC or RC and tracing of the contrast medium was performed to analyze distribution of injected cells. The experiment was designed to examine the distribution of the injected cells according to the methods of the invention.

Methods

Opensource software “3D Slicer” (version 5.0.3, www.slicer.org) was used to perform segmentation, modeling, visualization, simulation and measurements. Detection of injected KI was performed using automatic thresholding. In cases when KI was diluted with residual fluid inside inner ear after injection, the KI becomes harder to distinguish from the otic capsule and rest of the bones. In such cases, 3D scissor tool was used to edit the segmentation (remove bones). Subsequently, detected KI was volume calculated using segment statistics plug-in in the 3D slicer program and then categorized into different areas of the inner ear.

Results

Volumes of injected cells mixed with contrast medium differed between injected and traced. Many factors are behind this, such as “dead volume” in the needle, contrast medium further diluted with residual fluid inside inner ear, or water evaporating during scanning. These factors all play role during detection; therefore, segmentation was performed with emphasis to trace the distribution of injected cells/contrast medium and not the volume itself. Of course, if the detection threshold was changed, more volume would be detected.

(a) Bone 466000: Injection into IAC, 20 μL.

Segmentation was performed with emphasis to show correct distribution of injected cells/contrast medium. About 8 μL was detected and 99% distributed inside the therapeutic area defined as within cochlea (Rosenthal's canal RC, Osseus spiral lamina OSL and modiolus) and internal acoustic canal (IAC, exclude facial nerve and single nerve). Worth noting in this bone is the contrast medium diffusing from IAC into the RC all the way up to the apex (compare RC before and after injection). Detailed distribution is listed in Table 3.

TABLE 3

| | | | | | | | Non- | | |
|---|---|---|---|---|---|---|---|---|---|
| | | Cochlea | Non- | | | | therapeutic | SUM | Surg note |
| | Injection | Therapeutic | therapeutic | Vestibule | | IAC | Facial nerve/ | Detection | injection |
| # | site | RC/OSL/mod | ST/SV/SL | Ut/Sac/SCC | End-sac | Therapeutic | single nerve | μL | μL |
| 1 | IAC | 1.54619 | | 0.04456 | | 6.37935 | | 7.9701 | 20 |
| 2 | IAC | 3.0798 | | | | 10.974 | 13.4962 | 27.55 | 20 |
| 3 | IAC | 1.77779 | 1.92137 | 0.2326 | | 19.05924 | | 22.991 | 14 |
| 4 | IAC | | | | | 27.2207 | 4.5038 | 31.7245 | 20 |
| 5 | IAC | 1.1854 | 37.0792 | | | 2.3871 | | 40.6517 | 20 |
| 6 | IAC | | | | | 46.0008 | | 46.0008 | 20 |
| 7 | IAC | | | | | 5.7189 | | 5.7189 | 3.2 |
| 8 | IAC | | 0.24 | 0.362 | 1.6154 | 1.21915 | | 3.43655 | ? |
| 9 | RC | 1.95158 | 5.085524 | | | | | 7.03682 | 5 |
| 10 | RC | 4.61267 | 0.35419 | 0.08679 | | 1.86252 | | 6.91617 | 5 |

Table 3: Detailed distribution of detected cell/KI mixture in each anatomical structure in each bone.

TABLE 4

| | | | | | RC to IAC | | |
|---|---|---|---|---|---|---|---|
| | Injection | μL | % | | Or IAC to RC | | Control |
| # | site | Inside therapeutic area | | Intended | migra/diffus | Outside | SUM |
| 1 | IAC | 7.92554 | 99% | 80% | 19% | 1% | 100% |
| 2 | IAC | 14.0538 | 51% | 40% | 11% | 49% | 100% |
| 3 | IAC | 20.83703 | 91% | 83% | 8% | 9% | 100% |
| 4 | IAC | 27.2207 | 86% | 86% | 0% | 14% | 100% |
| 5 | IAC | 3.5725 | 9% | 6% | 3% | 91% | 100% |
| 6 | IAC | 46.0008 | 100% | 100% | 0% | 0% | 100% |
| 7 | IAC | 5.7189 | 100% | 100% | 0% | 0% | 100% |
| 8 | IAC | 1.21915 | 35% | 35% | 0% | 65% | 100% |
| 9 | RC | 1.95158 | 28% | 28% | 0% | 72% | 100% |
| 10 | RC | 6.47519 | 94% | 67% | 27% | 6% | 100% |
| | | Average | 69% | 62% | 7% | | |
| | | STDEV | 35% | 33% | 10% | | |

Table 4: Detected cell/KI mixture inside and outside therapeutic area presented in %.

(b) Bone 466001: Injection into IAC, 20 μL.

Worth noting here is the contrast medium diffusing from IAC into the RC all the way up to the apex (compare RC before and after injection). Detailed distribution is listed in Table 3. Difference is large portion of the cell/KI mixture was taken up by the facial nerve compared to 466000. Detected volume was larger as well, possibly because of residual volume in the temporal bone after washing. Other factors playing role may be position of the temporal bone during injection, position of the temporal bone while scanning and during bone transfer. Gravitation may contribute to KI diffusion into certain areas depending on temporal bone positioning.

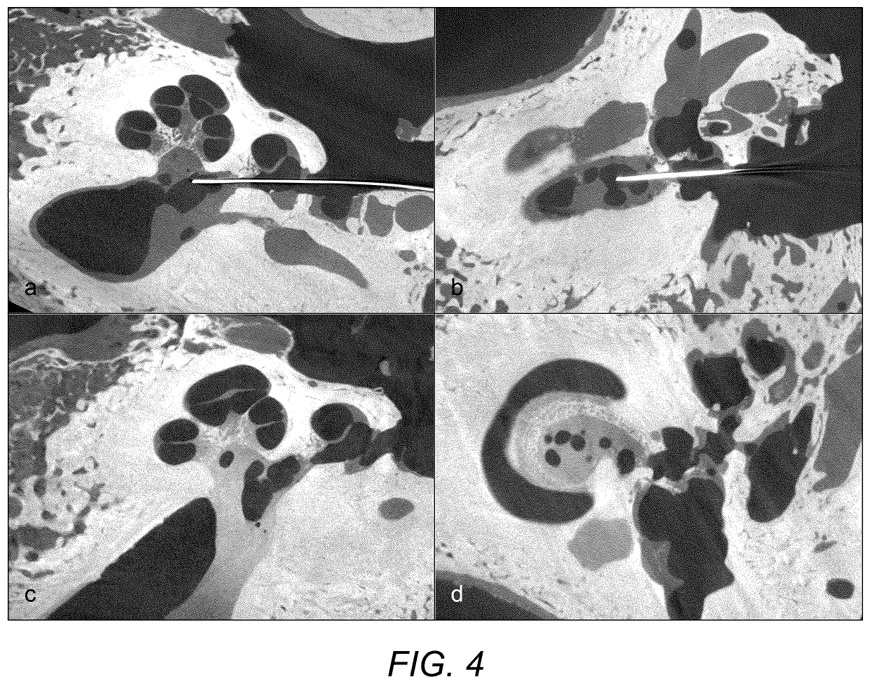

Confirmation that the cells were injected into the correct place within the temporal bone studies is shown with respect to the mages in FIG. 4 for bone 466001 on a 2D image. The upper two images show a drilled well and metal wire reaching into the IAC. The lower two images show the previous “dark nerve tissues” in the IAC became “white” since the tissues have taken up the contrast medium after injection. The Rosenthal's Canal in the lower images was seen to be whiter also and this was interpreted as the contrast medium having diffused through the neural tissues into the RC as well. FIG. 4 therefore shows that the technique of the invention allows injected cells to reach the right target.

(c) Bone 492000: Injection into IAC, 14 μL.

This bone is particularly hard, most of the cochlea were filled with liquid. KI taken up by the nerves could be identified after injection. In this bone, some of the KI was detected in the vestibule and inside cochlea. During segmentation, if the different structures have different pixel intensity, it is possible to distinguish the structures. Here the nerves inside IAC have similar pixel intensity as the bone after taken up KI, therefore manual removing bones (based on experience) was necessary after automatic thresholding.

(d) Bone 492001: Injection into IAC, 20 μL.

Most of the KI was taken up by the nerves inside IAC. Comparing to before injection, contrast medium/cells have migrated/diffused into the RC. However, comparing with the intensity of KI taken up in the nerves inside IAC, only very small fraction of KI diffused into RC. This small fraction was not able to be volume determined as threshold for detection for nearby structures are the same. It is most likely KI diffused into RC from IAC, as the scala tympani was clean from KI, so there were no back flush from injection. Significant amount of KI was taken up by the facial nerve. RC after injection in this bone is impossible to segment, as the pixel intensity is same as the bones nearby. Most KI was taken up by the nerves and automatic thresholding was set to trace the larger portion of KI inside IAC.

Discussion

An average of 69% injected cells/contrast medium reached therapeutic area, all bones and approaches were taken into account including practice bones. Of the 69% reaching to the therapeutic area, 7% were through diffusion.

Liquid remained in the cochlea after washing played important role for distribution. In previously operated bones, washing was not needed, and KI injected into the inner ear was easily detected and distribution pattern easily recognized to bones and soft tissues. In new temporal bones which required drilling and washing, the distribution pattern was more diffuse and probably more realistic. This also led to detected volumes in general were larger than injected.

### SUMMARY

In general, both injection methods were shown to be viable. IAC injection allows for high injection volume. This study therefore shows it is possible to deliver cells/contrast medium to a target area and trace them. Diffusion of contrast medium was observed within the therapeutic area from IAC to RC and from RC to IAC. These results confirm that the “inter-connected” spaces RC, osseus spiral lamina, IAC, Canalis cochlearis are in fact inter-connected. The results confirm previous studies in gerbils where otic progenitor cells migrated from IAC to RC. The study also confirms an injection volume of 20 μL.

### Example 3: Pilot Study for Administration of Cell-Based Treatment to Human Patients with Either Presbycusis or ANSD

A pilot study for the administration of a cell-based therapy for treatment of presbycusis or ANSD is as follows. Patients will be selected for suitability in the trial according to agreed criteria to receive the cell therapy in addition to unilateral cochlear implantation. Data will be collected at multiple timepoints: baseline, TO (day of surgery), T0+1 week, T0+1 month, T0+3 months, T0+6 months and T0+9 months, with additional daily self-testing of cochlear health from TO. Outcomes will relate to safety, cochlear health, speech outcomes and patient reported outcomes. Data in each group will be compared to historical controls.

Patients receiving the cell-based therapy will receive a single dose suspension, with a dosage of 0.15E+06 cells/ear. There will be no dose escalation. The cell suspension will be injected into the Canalis cochlearis (cochlear canal) and/or Rosenthal's Canal structures in the Internal Auditory Canal in the inner ear (cochlea) as described above as an adjunct to unilateral CI surgery. The injection will be administered during the CI surgery.

The primary endpoint of the study will be safety as measured by the frequency and severity of adverse events of cell-based therapeutic agent injection that are related to the drug substance, the procedure used to administer the drug substance, and/or any administered concomitant immunosuppression. Physical examination, safety imaging and standard ontological and audiological testing will be used to assess the safety.

Secondary endpoints may focus on objective measures of cochlear health and treatment efficacy:
assessment of spiral ganglion cell survival and function
assessment of auditory nerve function
audiometry and/or testing when there is sufficient residual hearing, and to assess the extent of residual acoustic hearing

Further exploratory endpoints may assess:
comfort and threshold hearing levels
speech perception testing
patient reported quality of life assessments

1. A method for the treatment of an auditory disease or condition in a human subject, comprising administering a therapeutic agent into a bony cavity of a cochlea of the subject that contains neural structures via the Round Window membrane of the cochlea.

2. The method of claim 1, wherein the auditory disease or condition is a hearing loss, deafness, or other auditory disorder associated with loss of inner ear function.

3. The method of claim 1, wherein the therapeutic agent is a composition comprising a pharmaceutically active substance or a composition comprising a population of cells.

4. The method of claim 3, wherein the therapeutic agent is a composition comprising a pharmaceutically active substance that is a compound, growth factor, hormone, or specific binding molecule.

5. The method of claim 4, wherein the pharmaceutically active substance is a compound that is an activator of inner ear progenitor cells or other molecule capable of stimulating inner ear progenitor cell growth.

6. The method of claim 4, wherein the pharmaceutically active substance is a compound that is an anticancer agent, an otoprotective agent, an antibiotic, or an anti-inflammatory agent.

7. The method of claim 4, wherein the pharmaceutically active substance is a growth factor is selected from the group consisting of insulin-like growth factor-1 (IGF-1), a or a steroid.

8. The method of claim 3, wherein the therapeutic agent is a population of cells that comprises fully- or partly-differentiated cells.

9. The method of claim 3, wherein the therapeutic agent is a population of cells that comprises auditory neurons, hair cell-like cells, spiral ganglion neurons, or sensory neurons.

10. The method of claim 3, wherein the therapeutic agent is a population of cells that comprises stem cells or progenitor cells.

11. The method of claim 1, wherein the bony cavity of the cochlea that contains neural structures is the Rosenthal's Canal or Canalis cochlearis.

12. (canceled)

13. (canceled)

14. A method for the delivery of a therapeutic agent to a cochlea of a human subject having an auditory disease or condition, comprising administering the therapeutic agent into a bony cavity of the cochlea of the subject that contains neural structures via the Round Window membrane of the cochlea.

15. The method of claim 14, wherein the bony cavity of the cochlea that contains neural structures is the Rosenthal's Canal or Canalis cochlearis.

16. The method of claim 10, wherein the therapeutic agent is a population of cells that comprises otic neural progenitor cells (ONPs).
